(19) United States
(12) Patent Application Publication
(10) Pub. No.: US 2017/0232011 A1
(43) Pub. Date: Aug. 17, 2017

(54) PHARMACEUTICAL COMPOSITION AND METHOD FOR THE TREATMENT OF URINARY TRACT INFECTIONS

(71) Applicants: Jill Sweazy, Columbia, SC (US); Scott Sweazy, Columbia, SC (US)

(72) Inventors: Jill Sweazy, Columbia, SC (US); Scott Sweazy, Columbia, SC (US)

(21) Appl. No.: 15/044,501

(22) Filed: Feb. 16, 2016

(51) Int. Cl. A61K 31/655; A61K 31/4178

(52) U.S. Cl. CPC A61K 31/655; A61K 31/4178

(57) ABSTRACT

A pharmaceutical composition and method for the treatment of urinary tract infections based on a combination of nitrofurantoin and an analgesic.

### BACKGROUND OF THE INVENTION

Urinary tract infection (UTI) caused by uropathogenic Escherichia coli (UPEC) is one of the most common infectious diseases afflicting mankind, particularly women. UTIs are the second most common reason for physician visits, and account for about 8.3 million doctor visits each year (Ambulatory Care Visits to Physician Offices, Hospital Outpatient Departments, and Emergency Departments: United States, 1999/2000. Vital and Health Statistics. Series 13, No. 157. Hyattsville, Md.: National Center for Health Statistics, Centers for Disease Control and Prevention, U.S. Dept. of Health and Human Services; September 2004.). The morbidity and economic impact of the disease are enormous, with over $2.5 billion spent annually on treatment. Furthermore, recurrent infections are a significant problem despite appropriate antibiotic therapy of the index case. The high rates of recurrence, and the large number of women who find themselves in urology clinics due to their chronic recurrent UTIs highlights the need for a better understanding of the pathogenic mechanisms involved in this disease and the development of new and better therapies.

It is an object of the present invention to provide a uniquely beneficial therapy regimen for urinary tract infections.

### SUMMARY OF THE INVENTION

The above and other objects are realized by the present invention, one embodiment of which relates to a pharmaceutical composition for the treatment of urinary tract infections comprising nitrofurantoin, an analgesic, and a pharmaceutically acceptable carrier therefore.

Another embodiment of the invention concerns a method for the treatment of urinary tract infections comprising the administration to a patient in need thereof an effective amount of a pharmaceutical composition comprising nitrofurantoin and an analgesic.

A still further embodiment of the invention comprises the above described pharmaceutical composition wherein the nitrofurantoin and analgesic are separately packaged.

An additional embodiment of the invention is a kit comprising in one or more containers, the above described pharmaceutical composition and instructions for using the contents thereof.

These and other features, aspects and advantages of the present invention will become better understood with reference to the following drawings, description and claims.

### BRIEF DESCRIPTION OF THE DRAWINGS

FIG. 1 is a view of the chemical structure of phenazopyridine.

FIG. 2 is a view of the chemical structure of nitrofurantoin.

FIG. 3 is a view of the chemical structure of nitrofurantoin monohydrate.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a pharmaceutical composition and method for the treatment of urinary tract infections based on a combination of nitrofurantoin and an analgesic. More particularly, the pharmaceutical composition of the invention comprises an amount of nitrofurantoin effective to treat urinary tract infections and an amount of analgesic, preferably phenazopyridine, effective to ameliorate the pain associated with urinary tract infections.

One of the difficulties encountered by health care workers in treating urinary tract infections is ensuring patient compliance with the recommended therapies. The uropathogenic bacteria responsible for urinary tract infections are extremely stubborn in that their successful eradication requires the chronic administration of effective antibiotics such as nitrofurantoin over extended periods of time. Many patients lose patience before their therapy has been completed and they abandon or fail to rigorously follow the dosing regimen prescribed for complete remission of the disease.

The present invention is predicated on the discovery that the co-administration of the antibiotic nitrofurantoin and an analgesic for the treatment of urinary tract infections results in an unexpected, significantly improved patient compliance with prescribed drug dosing regimens.

Without wishing to be bound by any theory or mechanism of action, it is believed that the alleviation of the pain associated with urinary tract infections by the analgesic co-administered with the antibiotic functions to relieve the anxiety suffered by patients over the length of treatment regimens, thereby ensuring better patient compliance.

The preferred antibiotic for inclusion in the composition of the invention is nitrofurantoin (1-[[[5-nitro-2-furanyl]methylene] amino]-2,4-imidazolidinedione). It is available in microcrystalline form (FIG. 2) or in the monohydrate form (FIG. 3). It is usually administered as a mixture of both forms. A typical dosage comprises a hard gelatin capsule shell containing the equivalent of 50-200 mg of nitrofurantoin in the form of 25% of nitrofurantoin macrocrystals and 75% of nitrofurantoin monohydrate. Typically, a patient will receive 2-3 dosages per day.

The efficacy of nitrofurantoin in treating UTIs combined with a low rate of bacterial resistance to this agent makes it one of the first-line agents for treating uncomplicated UTIs as recommended by the Infectious Diseases Society of America and the European Society for Microbiology and Infectious Diseases. Nitrofurantoin has been shown to have good activity against:

E. coli

Staphylococcus saprophyticus

Coagulase negative staphylococci

Enterococcus faecalis

Staphylococcus aureus

Streptococcus agalactiae

Citrobacter species

Klebsiella species

It is concentrated in the urine, leading to higher and more effective levels in the urinary tract than in other tissues or compartments. With a 100 mg oral dose, plasma levels are typically less than 1 μg/ml while in the urine it reaches 200 μg/ml.

The mechanism of action of nitrofurantoin is unique and complex. The drug works by damaging bacterial DNA, since its reduced form is highly reactive. This is made possible by the rapid reduction of nitrofurantoin inside the bacterial cell by flavoproteins (nitrofuran reductase) to multiple reactive intermediates that attack ribosomal proteins, DNA, respiration, pyruvate metabolism and other macromolecules within the cell. Nitrofurantoin exerts greater effects on bacterial cells than mammalian cells because bacterial cells activate the drug more rapidly. It is not known which of the actions of nitrofurantoin is primarily responsible for its bactericidal activity. The broad mechanism of action for this drug likely is responsible for the low development of resistance to its effects, as the drug affects many different processes important to the bacterial cell.

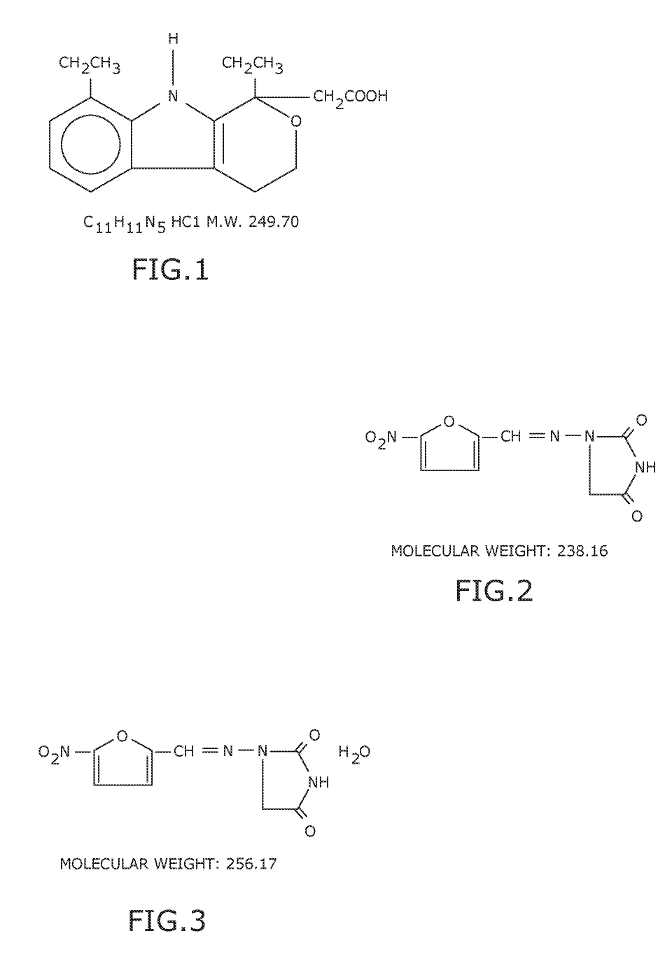

The preferred analgesic for co-administration with the antibiotic is phenazopyridine, 3-phenyldiazenylpyridine-2,6-diamine (FIG. 1). Phenazopyridine is usually administered orally in doses of 75 to 200 mg three times daily after meals.

Phenazopyridine is a chemical which, when excreted into the urine, has a local analgesic effect. It is often used to alleviate the pain, irritation, discomfort, or urgency caused by urinary tract infections, surgery, or injury to the urinary tract.

Phenazopyridine's mechanism of action is not well known, and only basic information on its interaction with the body is available. It is known that the chemical has a direct topical analgesic effect on the mucosa lining of the urinary tract. It is rapidly excreted by the kidneys directly into the urine. Hydroxylation is the major form of metabolism in humans, and the azo bond is usually not cleaved. On the order of 65% of an oral dose will be excreted directly into the urine chemically unchanged.

The combination of drugs may be combined in the same capsule or tablet or may be separately packaged and administered simultaneously.

It will be understood by those skilled in the art that any suitable pharmaceutically acceptable excipient/carrier material may be employed in compounding the compositions of the invention. Formulations of the present invention suitable for oral administration can be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug can also be administered in the form of a bolus, electuary or paste. Compressed tablets can be prepared by compressing, in a suitable machine, the drug in a free-flowing form such as a powder or granules, optionally mixed by a binder, lubricant, inert diluent, surface active or dispersing agent. Molded tablets can be made by molding, in a suitable machine, a mixture of the powdered drug and suitable carrier moistened with an inert liquid diluent.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A pharmaceutical composition for enhancing patient compliance with regimens for the treatment of urinary tract infections comprising nitrofurantoin, an analgesic, and a pharmaceutically acceptable carrier therefore.

2. The composition of claim 1 in tablet or capsule form.

3. The composition of claim 1 wherein said analgesic is phenazopyridine.

4. The composition of claim 1 in unit dosage form containing about 75 to 200 mg of nitrofurantoin and about 50 to 200 mg of analgesic.

5. The composition of claim 1 wherein the nitrofurantoin and analgesic are separately packaged.

6. The composition of claim 1 in kit form comprising nitrofurantoin and phenazopyridine in one or more containers, and instructions for using the contents thereof.

7. A method for enhancing patient compliance with regimen for the treatment of urinary tract infections comprising the administration to a patient in need thereof an effective amount of a pharmaceutical composition comprising nitrofurantoin and an analgesic.

8. The method of claim 6 comprising the administration of said pharmaceutical composition to a patient in need thereof three times daily.
